(19) United States
(12) Patent Application Publication
(10) Pub. No.: US 2011/0160265 A1
(43) Pub. Date: Jun. 30, 2011

(54) METHOD OF TREATING MOTOR DISORDERS WITH ALPHA-2B ADRENERGIC RECEPTOR AGONISTS

(76) Inventors: Lauren M.B. Luhrs, Rancho Santa Margarita, CA (US); Daniel W. Gil, Corona Del Mar, CA (US); John E. Donello, Dana Point, CA (US)

(21) Appl. No.: 12/595,191

(22) PCT Filed: Oct. 14, 2008
(86) PCT No.: PCT/US08/79775

§ 371 Date: Feb. 8, 2010

Related U.S. Application Data
(60) Provisional application No. 60/981,022, filed on Oct. 18, 2007.

(51) Int. Cl. A61K 31/4164; A61K 31/17; A61K 31/4174; A61K 31/4168; C07D 233/58; C07D 233/64; C07D 233/42; C07D 233/48; A61P 25/14

(52) U.S. Cl. 514/398; 514/587; 514/396; 548/335.1; 548/344.1; 548/325.1; 548/331.5

(57) ABSTRACT

Disclosed herein is a method of treating motor disorders comprising administering to a subject in need of such treatment an alpha-2 receptor agonist lacking significant alpha-2A receptor activity.

### CROSS-REFERENCE

This application claims the benefit of U.S. Application Ser. No. 60/981,022, filed Oct. 18, 2007, which is hereby incorporated by reference in its entirety.

Disclosed herein is a method of treating motor disorders by administering to a subject an alpha-2 adrenergic receptor agonist lacking significant alpha-2A receptor activity. Such agonists are effective in treating the disorders without sedating the patient to whom they are administered.

### DETAILED DESCRIPTION OF THE INVENTION

### Alpha-2 Receptor Agonists Lacking Significant Alpha-2A Activity

Alpha-2 receptor agonists are those compounds that activate alpha-2 adrenergic receptors. There are three subtypes of this receptor, designated A, B, and C. A compound is an “alpha-2B receptor agonist” if it has greater than 25% efficacy relative to brimonidine at the alpha-2B adrenergic receptor; a compound is an “alpha-2C receptor agonist” if it has greater than 25% efficacy relative to brimonidine at the alpha-2C adrenergic receptor; and a compound is an “alpha-2B/2C receptor agonist” if it has greater than 25% efficacy relative to brimonidine at both the alpha-2B and alpha-2C adrenergic receptors.

The methods of the present invention use alpha-2 agonists lacking significant activity at the alpha-2A receptor subtype. An agonist lacks significant alpha-2A receptor activity if the agonist has less than 40% of the efficacy of brimonidine at the alpha-2A receptor subtype. Compounds of the invention include, therefore, alpha-2B receptor agonists lacking significant alpha-2A activity; alpha 2B/2C receptor agonists lacking significant alpha-2A activity; and alpha-2C receptor agonists lacking significant alpha-2A activity. Any of the foregoing compounds may be used, even if they bind receptors other than alpha-2 receptors; for example, alpha-1 receptor agonists may be used, provided that the alpha-1 agonists also have greater than 25% efficacy relative to brimonidine at one or both of the alpha-2B and alpha-2C receptor subtypes, and lack significant alpha-2A receptor activity.

Efficacy, also known as intrinsic activity, is a measure of maximal receptor activation achieved by a compound and can be determined using any accepted assay of alpha-adrenergic receptor activation, such as a cAMP or Receptor Selection and Amplification Technology (RSAT). Efficacy is represented as a ratio or percentage of the maximal effect of the drug to the maximal effect of a standard agonist for each receptor subtype. Brimonidine, itself an alpha-2B receptor agonist (it is has 100% the efficacy of brimonidine at the alpha-2B adrenergic receptor), is used as the standard agonist for the alpha-2B adrenergic receptors.

Agonist activity can be characterized using any of a variety of routine assays, including, for example, Receptor Selection and Amplification Technology (RSAT) assays (Messier et al., Pharmacol. Toxicol. 76:308-11 (1995); cyclic AMP assays (Shimizu et al., J. Neurochem. 16:1609-1619 (1969)); and cytosensor microphysiometry assays (Neve et al., J. Biol. Chem. 267:25748-25753 (1992)). Such assays generally are performed using cells that naturally express only a single alpha-adrenergic receptor subtype, or using transfected cells expressing a single recombinant alpha-adrenergic receptor subtype. The adrenergic receptor can be a human receptor or homolog of a human receptor having a similar pharmacology.

The RSAT assay measures receptor-mediated loss of contact inhibition resulting in selective proliferation of receptor-containing cells in a mixed population of confluent cells. The increase in cell number is assessed with an appropriate detectable marker gene such as beta-galactosidase, if desired, in a high throughput or ultra high throughput assay format. Receptors that activate the G protein, Gq, elicit the proliferative response. Alpha-adrenergic receptors, which normally couple to Gi, activate the RSAT response when coexpressed with a hybrid Gq protein containing a Gi receptor recognition domain, designated Gq/i5. Conklin et al., Nature 363:274-6 (1993)).

As an example, an RSAT assay can be performed essentially as follows. NIH-3T3 cells are plated at a density of 2×10⁶ cells in 15 cm dishes and maintained in Dulbecco's modified Eagle's medium supplemented with 10% calf serum. One day later, cells are cotransfected by calcium phosphate precipitation with mammalian expression plasmids encoding p-SV-β-galactosidase (5-10 μg), receptor (1-2 μg) and G protein (1-2 μg). Carrier DNA, for example 40 μg salmon sperm DNA, also can be included to increase transfection efficiency. Fresh media is added on the following day; one to two days later, cells are harvested and frozen in 50 assay aliquots. Transfected cells are thawed, and 100 μl of cells added to 100 μl aliquots of compound to be tested, with various concentrations assayed in triplicate, for example, in 96-well plates. Incubation continues for 72 to 96 hours at 37° C. After washing with phosphate-buffered saline, β-galactosidase activity is determined by adding 200 μl of chromogenic substrate (3.5 mM O-nitrophenyl-β-D-galactopyranoside/0.5% NP-40 in phosphate buffered saline), incubating overnight at 30° C., and measuring optical density at 420 nm. The absorbancy is a measure of enzyme activity, which depends on cell number and reflects receptor-mediated cell proliferation. The EC₅₀ and maximal effect (i.e., efficacy) of each drug at each receptor is determined.

Alpha-2B and -2C receptor agonists lacking significant alpha-2A receptor activity are known in the art. Detailed information regarding alpha-2 agonists, including their structure, synthesis, and activity, may be found in U.S. Pat. No. 6,329,369, U.S. Pat. No. 6,534,542, U.S. Pat. No. 6,545,182, U.S. Pat. No. 6,787,517, U.S. Pat. No. 6,841,684, and U.S. Pat. No. 7,091,232; in U.S. Patent Application Publication No. 2003/0092766, No. 2004/0132824, No. 2004/0220402, No. 2005/0075366, and No. 2005/0267186; and in U.S. patent application Ser. No. 11/172,229, Ser. No. 11/232,323, Ser. No. 11/232,341, No. 60/613,870, No. 60/695,650, No. 60/747,444, No. 60/884,718, No. 60/917,828, No. 60/911,422, No. 60/911,478, and No. 60/948,389, the disclosures of all which are incorporated herein by reference.

One can use in the methods of the invention any pharmaceutically acceptable salt, prodrug, isomer, or racemate of any alpha-2 receptor agonist lacking significant alpha-2A receptor activity.

### Pharmaceutically Acceptable Salts

Alpha-2 receptor agonists may be used as their pharmaceutically acceptable salts.

A “pharmaceutically acceptable salt” is any salt that retains the activity of the parent compound and does not impart any additional deleterious or untoward effects on the subject to which it is administered and in the context in which it is administered compared to the parent compound. A pharmaceutically acceptable salt also refers to any salt which may form in vivo as a result of administration of an acid, another salt, or a prodrug which is converted into an acid or salt.

Pharmaceutically acceptable salts of acidic functional groups may be derived from organic or inorganic bases. The salt may comprise a mono or polyvalent ion. Of particular interest are the inorganic ions lithium, sodium, potassium, calcium, and magnesium. Organic salts may be made with amines, particularly ammonium salts such as mono-, di- and trialkyl amines or ethanol amines. Salts may also be formed with caffeine, tromethamine and similar molecules. Hydrochloric acid or some other pharmaceutically acceptable acid may form a salt with a compound that includes a basic group, such as an amine or a pyridine ring.

### Prodrugs

One can use in the compositions and methods of the invention a prodrug of any alpha-2 receptor agonist.

A “prodrug” is a compound which is converted to a therapeutically active compound after administration, and the term should be interpreted as broadly herein as is generally understood in the art. While not intending to limit the scope of the invention, conversion may occur by hydrolysis of an ester group or some other biologically labile group. Generally, but not necessarily, a prodrug is inactive or less active than the therapeutically active compound to which it is converted. Ester prodrugs of the compounds disclosed herein are specifically contemplated. An ester may be derived from a carboxylic acid of C1 (i.e., the terminal carboxylic acid of a natural prostaglandin), or an ester may be derived from a carboxylic acid functional group on another part of the molecule, such as on a phenyl ring. While not intending to be limiting, an ester may be an alkyl ester, an aryl ester, or a heteroaryl ester. The term alkyl has the meaning generally understood by those skilled in the art and refers to linear, branched, or cyclic alkyl moieties. C₁₋₆ alkyl esters are particularly useful, where alkyl part of the ester has from 1 to 6 carbon atoms and includes, but is not limited to, methyl, ethyl, propyl, isopropyl, n-butyl, sec-butyl, iso-butyl, t-butyl, pentyl isomers, hexyl isomers, cyclopropyl, cyclobutyl, cyclopentyl, cyclohexyl, and combinations thereof having from 1-6 carbon atoms, etc.

The alpha-2 receptor agonists of the invention may be either synthetically produced, or may be produced within the body after administration of a prodrug. Hence, the term “alpha-2 receptor agonist” encompasses both compounds produced by a manufacturing process and those compounds formed in vivo only when another drug administered.

### Isomers and Racemates

One can use in the compositions and methods of the invention an enantiomer, stereoisomer, or other isomer of an alpha-2 receptor agonist. One can also use in the compositions and methods of the invention a racemic mixture or one or both racemates, in any proportion.

### Dose

The precise dose and frequency of administration depends on the severity and nature of the patient's condition, on the manner of administration, on the potency and pharmacodynamics of the particular compound employed, and on the judgment of the prescribing physician. Determining dose is a routine matter that is well within the capability of someone of ordinary skill in the art. In general, alpha-2 receptor agonists are administered in therapeutically effective doses, that is, at a dose that is sufficient to produce the desired therapeutic effect.

### Excipients and Dosage Forms

Those skilled in the art will readily understand that alpha-2 receptor agonists can be admixed with pharmaceutically acceptable excipients which are well known in the art.

A pharmaceutical composition to be administered systemically may be confected as a powder, pill, tablet or the like, or as a solution, emulsion, suspension, aerosol, syrup or elixir suitable for oral or parenteral administration or inhalation.

For solid dosage forms or medicaments, non-toxic solid carriers include, but are not limited to, pharmaceutical grades of mannitol, lactose, starch, magnesium stearate, sodium saccharin, the polyalkylene glycols, talcum, cellulose, glucose, sucrose and magnesium carbonate. The solid dosage forms may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the technique described in U.S. Pat. No. 4,256,108, U.S. Pat. No. 4,166,452, and U.S. Pat. No. 4,265,874 to form osmotic therapeutic tablets for control release. Liquid pharmaceutically administrable dosage forms can, for example, comprise a solution or suspension of one or more of the presently useful compounds and optional pharmaceutical adjutants in a carrier, such as for example, water, saline, aqueous dextrose, glycerol, ethanol and the like, to thereby form a solution or suspension. If desired, the pharmaceutical composition to be administered may also contain minor amounts of nontoxic auxiliary substances such as wetting or emulsifying agents, pH buffering agents and the like. Typical examples of such auxiliary agents are sodium acetate, sorbitan monolaurate, triethanolamine, sodium acetate, triethanolamine oleate, etc. Actual methods of preparing such dosage forms are known, or will be apparent, to those skilled in this art; for example, see Remington's Pharmaceutical Sciences, Mack Publishing Company, Easton, Pa., 16th Edition, 1980. The composition of the formulation to be administered, in any event, contains a quantity of one or more of the presently useful compounds in an amount effective to provide the desired therapeutic effect.

Parenteral administration is generally characterized by injection, either subcutaneously, intramuscularly or intravenously. Injectables can be prepared in conventional forms, either as liquid solutions or suspensions, solid forms suitable for solution or suspension in liquid prior to injection, or as emulsions. Suitable excipients are, for example, water, saline, dextrose, glycerol, ethanol and the like. In addition, if desired, the injectable pharmaceutical compositions to be administered may also contain minor amounts of non-toxic auxiliary substances such as wetting or emulsifying agents, pH buffering agents and the like.

### Motor Disorders

Compounds of the invention are useful in treating motor disorders. To “treat,” as used here, means to deal with medically. It includes administering an alpha-2B receptor agonist to prevent the onset of a condition, to diminish its severity, and to prevent its reoccurrence. The inventors have discovered that the compounds of the invention may be used to treat motor disorders without causing the sedation that ordinarily accompanies the administration of alpha-2 agonists.

A “motor disorder,” as that term is used here, is any condition in which a subject experiences involuntary, undesirable movements that are independent of any deficits in sensorimotor gating; that is, the movement is not the result of abnormal motor output in response to sensory input information.

In one embodiment, the motor disorder is mediated by changes (for example, an increase or a decrease) in the availability or utilization of dopamine in the nervous system; hence, compounds of the invention may be used to treat motor disorders associated with hyper- or hypo-dopamine conditions of the nervous system.

Exemplary motor disorders which may be treated with the compounds of the invention include, for example, L-Dopa-induced dyskinesias, tardive dyskinesias, cervical dystonia, spinal torticollis, blepharospasm/Meige's disease, restless leg syndrome, essential tremor, rigidity (Parkinson's disease-associated or otherwise specified), ataxic disorder, spasticity.

### EXAMPLES

The invention is illustrated by the following examples. This is provided for illustration only; many more embodiments are possible.

Alpha-2B receptor agonists were evaluated in tests of amphetamine-induced hyperlocomotion, cocaine-induced hyperlocomotion, and nigrostriatal-lesion-induced rotational behavior following amphetamine administration. Six different compounds and four different alpha-2B selective pharmacophores were used: Compound A (thiourea), Compounds B and C (imidazoles), Compounds D and E (imidazole thiones), and Compound F (amino imidazoline), all of which lack significant alpha-2A receptor activity.

### General Findings

Amphetamine-induced and cocaine-induced hyperactivity are models of increased dopamine-mediated locomotion. The nigrostriatal lesion-induced rotation model exploits the imbalance in dopaminergic innervation following a unilateral lesion of the dopamine-containing substantia nigra pars compacta (SNc), thereby creating a hemiparkinsonian condition. The subsequent rotational behavior in these animals induced by various drugs can be used to infer the level of dopamine imbalance on the lesioned and unlesioned side of the brain. In amphetamine- and cocaine-induced hyperactivity, Compounds A-F were able to effectively inhibit increased locomotion associated with psychostimulant administration. In addition, these compounds were also able to inhibit amphetamine-induced rotational behavior in nigrostriatal-lesioned animals.

Importantly, these compounds are orally active, and therefore could be administered in solution, tablet or capsule.

Table 1, below, shows the structures of Compounds A-F:

| COMPOUND | COMPOUND STRUCTURE |
|---|---|
| A | 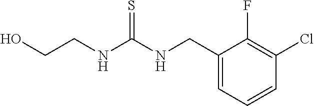 |
| B | 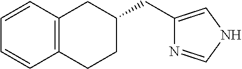 |
| C | 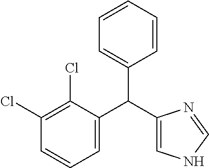 |
| D | 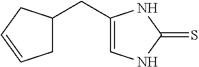 |
| E | 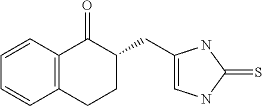 |
| F | 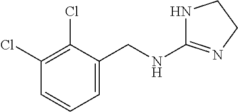 |

### Amphetamine-Induced Hyperlocomotion

Table 2, below, indicates the locomotor responses of mice treated with various combinations of alpha-2B receptor agonists and the psychostimulant amphetamine. Drug 1 was administered 30 minutes after the animal was placed in the open field. Drug 2 was administered subcutaneously (SC) at 45 minutes after the animal was placed in the open field (15 minutes after drug 1). AUC indicates the total amount of locomotor activity after administration of drug 2. “% decrease activity” indicates the percentage decrease in total activity relative to the Vehicle+Amphetamine group. ° indicates significant difference (P<0.05) relative to Vehicle+Amphetamine treated animals. * indicates significant difference (P<0.05) relative to Vehicle+Vehicle treated animals.

| | | | | TOTAL ACTIVITY | % DECREASE |
|---|---|---|---|---|---|
| | | | | POST- | ACTIVITY REL |
| | DOSE | | DRUG 2 | AMPHETAMINE | TO |
| DRUG 1 | (MG/KG) | ROUTE | (SC) | (AUC) | VEH + AMPH |
| Vehicle | 0 | IP | Vehicle | 3328.83 ± 824.0 | 73% ^{⋄} |
| Vehicle | 0 | IP | Amph | 12291 ± 1796.6 | n/a |
| Haloperidol | 0.2 | IP | Amph | 1123.2 ± 280.1 | 91% ^{⋄} * |
| Compound A | 0.03 | IP | Amph | 5524.8 ± 624.2 | 55% ^{⋄} |
| Compound B | 0.03 | IP | Amph | 3940.5 ± 573.7 | 68% ^{⋄} |
| Compound B | 1 | IP | Amph | 4462.0 ± 957.5 | 64% ^{⋄} |
| Compound D | 0.01 | PO | Amph | 10664.0 ± 786.2 | 14% * |
| Compound D | 0.03 | PO | Amph | 5987.5 ± 1369. | 51% ^{⋄} |
| Compound D | 0.1 | PO | Amph | 6992.5 ± 693.1 | 43% ^{⋄} * |
| Compound D | 1 | PO | Amph | 4650.0 ± 628.9 | 62% ^{⋄} |
| Compound F | 0.03 | IP | Amph | 9933.0 ± 1898.7 | 26% ^{⋄} * |
| Compound F | 0.3 | IP | Amph | 9442.3 ± 1306.7 | 29% ^{⋄} * |
| Compound F | 1 | IP | Amph | 7437.8 ± 1812.4 | 45% ^{⋄} |
| Compound F | 3 | IP | Amph | 5169.0 ± 1481.4 | 62% ^{⋄} |
| Compound E | 0.01 | PO | Amph | 11419.2 ± 550.4 | 15% * |
| Compound E | 0.03 | PO | Amph | 5616.8 ± 618.2 | 58% ^{⋄} |
| Compound E | 0.1 | PO | Amph | 4623.8 ± 1166.7 | 66% ^{⋄} |
| Compound E | 1 | PO | Amph | 6867.3 ± 979.3 | 49% ^{⋄} * |
| Compound E | 10 | PO | Amph | 6026.0 ± 1007.9 | 55% ^{⋄} |
| Compound C | 0.03 | IP | Amph | 6348.0 ± 423.4 | 52% ^{⋄} |

### Cocaine-Induced Hyperlocomotion

Table 3, below, indicates the locomotor responses of mice treated with various combinations of alpha-2B receptor agonists and the psychostimulant cocaine. Drug 1 was administered 30 minutes after the animal was placed in the open field. Drug 2 was administered subcutaneously (SC) at 45 minutes after the animal was placed in the open field (15 minutes after drug 1). AUC indicates the total amount of locomotor activity after administration of drug 2. “% decrease activity” indicates the percentage decrease in total activity relative to the Vehicle+Cocaine group. ° indicates significant difference (P<0.05) relative to Vehicle+Cocaine treated animals. * indicates significant difference (P<0.05) relative to Vehicle+Vehicle treated animals.

| | | | | TOTAL ACTIVITY | |
|---|---|---|---|---|---|
| | DOSE | | DRUG 2 | POST-COCAINE | % DECREASE |
| DRUG 1 | (MG/KG) | ROUTE | (SC) | (AUC) | ACTIVITY |
| Vehicle | 0 | PO | Vehicle | 2861.2 ± 497.33 | 83% ^{⋄} |
| Vehicle | 0 | PO | Cocaine | 10391 ± 1208.32 | n/a |
| Compound A | 0.03 | PO | Cocaine | 8469 ± 935.01 | 19% * |
| Compound A | 0.1 | PO | Cocaine | 6457.67 ± 754.0 | 38% ^{⋄} * |
| Compound A | 0.3 | PO | Cocaine | 3543 ± 648.02 | 66% ^{⋄} |
| Compound E | 0.01 | PO | Cocaine | 5036.5 ± 464.2 | 52% ^{⋄} * |
| Compound E | 0.03 | PO | Cocaine | 5033.5 ± 503.8 | 52% ^{⋄} * |
| Compound E | 0.1 | PO | Cocaine | 6263.0 ± 436.6 | 40% ^{⋄} * |
| Compound E | 0.3 | PO | Cocaine | 3662.1 ± 1458.2 | 65% ^{⋄} |

### Nigrostriatal-Lesion-Induced Rotational Behavior Following Amphetamine Administration

Table 4, below, indicates the changes in rotational behavior in nigrostriatal-lesioned rats treated with various alpha-2B receptor agonists and the psychostimulant amphetamine. Drug 1 was administered 15 minutes before amphetamine, and 10 minutes post-amphetamine, the animals were placed into the rotometer for 30 or 60 minutes. “% decrease rotations” indicates the percentage decrease in total rotations relative to the Vehicle+Amphetamine group. ^{⋄} indicates significant difference (P<0.05) relative to Vehicle+Amphetamine treated animals. * indicates significant difference (P<0.05) relative to Vehicle+Vehicle treated animals.

| | DOSE | | DRUG 2 | % DECREASE |
|---|---|---|---|---|
| DRUG 1 | (MG/KG) | ROUTE | (SC) | ROTATIONS |
| Vehicle | 0 | PO | Vehicle | 91% ^{⋄} |
| Vehicle | 0 | PO | Amph | n/a |
| Compound A | 0.03 | PO | Amph | 65% ^{⋄} * |
| Compound B | 0.03 | PO | Amph | 29% * |
| Compound D | 0.1 | PO | Amph | 51% ^{⋄} * |
| Compound D | 1 | PO | Amph | 60% ^{⋄} * |
| Compound E | 0.3 | PO | Amph | 58% ^{⋄} * |

### Materials and Methods

Amphetamine- or cocaine-induced hyperactivity. Mice were placed in an open field apparatus (FlexField, San Diego Instruments, San Diego, Calif.). After 30 minutes of habituation, they received a vehicle or haloperidol injection or injection of alpha-2B receptor agonist followed by an injection of amphetamine (2 mg/kg, s.c.) or cocaine (10 mg/kg, i.p.) at minute 45. Their activity levels were subsequently measured for another 1 hour post-injection. Total activity over each 5 minute bin (21 total bins) was added to establish activity curve over the 105 minute testing.

Amphetamine-induced rotational behavior. Pre-Apomorphine-screened (rotated) rats (with lesions ≧95% DA cell loss) were purchased from Charles Rivers Laboratories. Animals were given ˜4 days to acclimate prior to testing. All animals weighted 250-300 g at the beginning of the study. Animals were treated with various combinations of vehicle or alpha-2B receptor agonist and amphetamine.

Vehicle or alpha-2B receptor agonist was administered (p.o.) 15 minutes before amphetamine (2 mg/kg s.c.). Rotational behavior was assessed 15 minutes post-amphetamine administration and was monitored for 60 minutes using a Rotometer system from San Diego Instruments (San Diego, Calif.).

For i.p. administration, the compounds are formulated in H₂O with 0.5% DMSO and given in a volume of 1 ml/kg body weight by injecting into the intraperitoneal cavity. For p.o. administration, the compounds are formulated in H₂O with 0.5% DMSO and given in a volume of 2 ml/kg body weight using a 25-gauge, 1.5 inch gavage needle that is slowly inserted through the esophagus into the stomach.

1. A method of treating a motor disorder, the method comprising administering to a subject in need of such treatment an alpha-2 receptor agonist lacking significant alpha-2A receptor activity.

2. The method of claim 1, wherein the method further comprises treating the motor disorder without causing sedation.

3. The method of claim 2, wherein the alpha-2 receptor agonist is selected from the group consisting of an alpha-2B receptor agonist and an alpha-2B/2C receptor agonist.

4. The method of claim 2, wherein the alpha-2 receptor agonist is an alpha-2C receptor agonist.

5. The method of claim 2, wherein the motor disorder is associated with a hyper- or hypo-dopamine condition of the nervous system.

6. The method of claim 5, wherein the disorder is selected from the group consisting of dystonia, L-Dopa-induced dyskinesias, tardive dyskinesias, cervical dystonia, spinal torticollis, blepharospasm/Meige's disease, restless leg syndrome, essential tremor, rigidity (Parkinson's disease-associated or otherwise specified), ataxic disorder, and spasticity.

7. The method of claim 6, wherein the alpha-2B receptor agonist is a compound having the following structure:

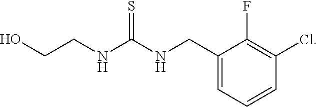

8. The method of claim 6, wherein the alpha-2B receptor agonist is a compound having the following structure:

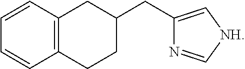

9. The method of claim 6, wherein the alpha-2B receptor agonist is a compound having the following structure:

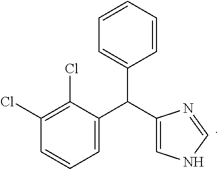

10. The method of claim 6, wherein the alpha-2B receptor agonist is a compound having the following structure:

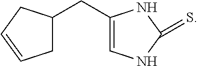

11. The method of claim 6, wherein the alpha-2B receptor agonist is a compound having the following structure:

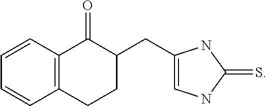

12. The method of claim 6, wherein the alpha-2B receptor agonist is a compound having the following structure:

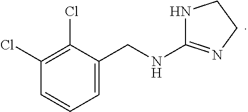

13. A compound selected from the group consisting of: 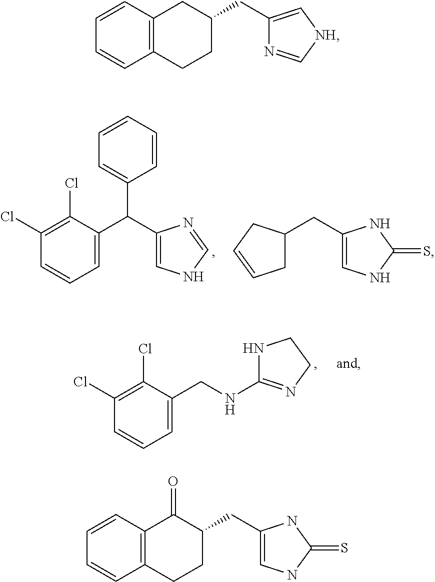
including its alternate solid forms, tautomers, stereoisomers, enantiomers, diastereomers, prodrugs, and pharmaceutically acceptable salts, hydrates and solvates.

14. The compound of claim 13 wherein the compound is selected from the group consisting of 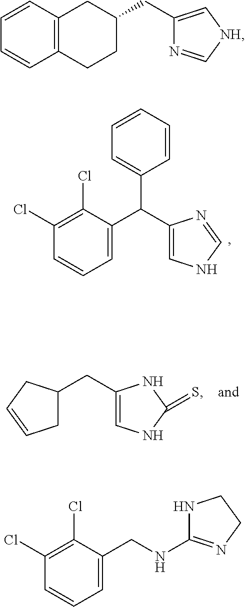
and pharmaceutically acceptable salts.

15. The compound of claim 13 wherein the compound is [chemical structure]
